(19) United States
(12) Patent Application Publication
(10) Pub. No.: US 2011/0021639 A1
(43) Pub. Date: Jan. 27, 2011

(54) Microbially stable dispersion medium for emulsions

(75) Inventors: James Paik, Chicago, IL (US); Howard Haley, Three Rivers, MI (US)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: KALAMAZOO HOLDINGS, INC.

(21) Appl. No.: 12/804,401

(22) Filed: Jul. 21, 2010

Related U.S. Application Data
(60) Provisional application No. 61/271,545, filed on Jul. 22, 2009.

(51) Int. Cl. A61K 31/045; A23L 1/222

(52) U.S. Cl. 514/729; 426/651

(57) ABSTRACT

The present invention relates to novel microbial stable emulsions advantageously utilizing dispersion media characterized by lower water activity and advantageously utilizing dispersants which exhibit high water binding capacity and which exhibit characteristics which provide for steric stabilization and charge distribution (zeta potential) in relation to the dispersed phase, and methods of using the same.

### FIELD OF THE INVENTION

The present invention relates to novel microbial stable emulsions advantageously utilizing dispersion media characterized by lower water activity and advantageously utilizing dispersants which exhibit high water binding capacity and which exhibit characteristics which provide for steric stabilization and charge distribution (zeta potential) in relation to the dispersed phase, and methods of using the same.

### BACKGROUND OF THE INVENTION

An emulsion is commonly defined as a mixture of one or more immiscible substances, generally in liquid form. Typically, emulsions comprise two immiscible liquid phases that comprise dispersion media and dispersed phase. Emulsions require some form of mechanical energy to reduce particle size of dispersed particles and emulsions may be characterized by the stability of this mixture or its tendency to revert back to its constituent phases.

At the very least, emusions may typically comprise dispersants such as steric stabilizers, which provide for emulsion stability. Steric stabilizers are polymers that are adsorbed or attach to the surface of the dispersed phase. These polymers prevent aggregation of dispersed phase particles by preventing the dispersed phase particles from forming large particles through van der Waals attraction. The attached polymers physically repel particles attracted by van der Waals force. The steric stabilizing capability of these polymers depends on their specific conformation. The polymer conformation exhibits hydrophobic or hydrophilic domains which will adsorb on the surface of dispersed phase particles and extend to form a physical barrier to distance the dispersed phase particles. Polymers are selected for their conformation as such conformation relates to the dispersed phase and any solvents.

Biopolymer steric stabilizers from natural materials usually exhibit conformation suitable for use with water based solvents. However, steric stabilizer performance may be compromised when other solvents such as glycerol or ethanol are added to the dispersion medium.

Zeta potential is another factor that is important in emulsion stability. The electric potential at the surface of hydrodynamic shear is the zeta potential. Zeta potential is a measure of the electrical charge stabilization of an emulsion system and will depend on the composition of dispersion medium.

Emulsion technology finds application in numerous products, such as paints, cosmetics, pharmaceuticals, and food.

Emulsion technology is selected for formulating a variety of active and inactive substances because emulsions make it possible to deliver active substances which may not otherwise be soluble in the form of a solution. One common method for delivering liquid hydrophobic actives as dispersed particles in aqueous emulsions is to dissolve a substantial amount (20-50%) of surfactants in the dispersion medium.

In addition to an emulsion's tendency to revert to its constituent phases, emulsions comprising an aqueous phase are susceptible to microbial growth. Numerous techniques may be employed to treat or prevent microbial infestation in emulsions. For example, it is common practice to add antimicrobial agents to aqueous emulsions. It is a common practice to use antimicrobial agents in pharmaceutical emulsions and food emulsions. In food, very mild antimicrobial agents such as benzoic acid, nitrites, and sulfites are used.

Clearly, there are emulsions for which antimicrobial treatment may not be desirable. For example, artificial antimicrobial agents cannot be used in foods labeled as “all natural”. Similarly, antimicrobial agents imparting undesirable taste or odor may not find application in foods.

An alternative to the use of artificial antimicrobial agents is to make water less available for microbial growth. It is well documented that microorganisms cannot grow when water is not available. It has been determined that water activity rather than the amount of water in solution determines growth of microorganisms. SCOTT, WJ. Water relations of Staphylococcus aureus at 30 degrees C., Aust J Biol Sci. 1953 Nov; 6(4):549-564. For example, microorganisms are unable to grow in honey which has about 60% moisture content. However, microorganisms can grow in bread which has about 20% moisture content. This is because the enthalpy of mixing between water and fructose/glucose in honey is much higher than water and starch in bread.

The term water activity was created by food scientists. It is defined as the partial pressure of the solution divided by partial pressure of pure water at a given temperature. The greater the enthalpy of mixing, the lower the partial pressure of water for a given solution. Water activity required for microbial growth is approximately between 0.61-0.99 depending on the microbial species and environment. Minimum ranges of water activity for growth of microrangisms are 0.95-0.91 for bacteria, 0.91-0.87 for yeasts, and 0.87-0.61 for molds. The water activity is typically controlled by adding solutes which have varying enthalpy of mixing. Practically speaking, shelf life of food may be adjusted by providing solutes that bind more strongly to water. The enthalpy of mixing is the excess enthalpy that determines solubility and binding energy between solute and solvent.

### THE PRESENT INVENTION

We have discovered novel microbial stable emulsions. The instant novel emulsions utilize dispersion media characterized by lower water activity, which characteristic provides for microbial stability without the addition of artificial antimicrobials, which antimicrobials may not be suitable for some use environments. Therefore, the instant emulsions find application in products which may be labeled “all natural” or “without preservatives”. In addition to providing for microbial stability through their low water activity, the instant dispersion media are optimized to favorably interact with dispersants which exhibit high water binding capacity and which exhibit characteristics which provide for steric stabilization and charge distribution (zeta potential) vis-à-vis the dispersed phase, thereby providing for emulsion stability.

### OBJECTS OF THE INVENTION

It is an object of the present invention to provide novel microbial stable emulsions not comprising antimicrobial agents. It is a further object to provide a novel method of stabilizing emulsions by optimizing the dispersion media to favorably interact with dispersants. It is a further object to provide for such novel emulsions further comprising dispersants such as, for example, natural biopolymers which function as a steric stabilizers. It is an object of the invention to provide such novel emulsions utilizing dispersion media which optimally interact with such dispersants by means of their characteristic water activity below 0.95 and a charge distribution (zeta potential) which supports emulsion stability.

Yet additional objects will become apparent hereinafter, and still further objects will be apparent to one skilled in the art.

### SUMMARY OF THE INVENTION

What we therefore believe to be comprised by our invention may be summarized inter alia in the following words:

A microbial stable emulsion comprising dispersion media characterized by lower water activity, one or more dispersants characterized by higher water binding capacity, and a lipophilic dispersed phase, wherein the dispersion media is present at a concentration below that in which microorganisms may grow in the emulsion;

Such a microbial stable emulsion which does not comprise an antimicrobial agent;

Such a microbial stable emulsion which is characterized by being resistant to microorganism contamination, wherein such microorganism contamination is selected from bacterial, yeast and fungal contamination;

Such a microbial stable emulsion, which is characterized by being resistant to microbial growth for a period exceeding twelve months;

Such a microbial stable emulsion, which is characterized by being resistant to microbial growth for a period exceeding six months;

Such a microbial stable emulsion, which is characterized by being resistant to microbial growth for a period exceeding four months;

Such a microbial stable emulsion, wherein the dispersion media may be characterized by a water activity below 0.95;

Such a microbial stable emulsion, wherein the dispersion media comprises one or more small molecular weight carbohydrates, water, ethanol;

Such a microbial stable emulsion, wherein the dispersion media small molecular weight carbohydrate may be selected from glycerol, sugar, sugar alcohols and glucose oligomers selected from the group consisting of sucrose, glucose, fructose, maltose, lactose, galactose, xylose, xylitol, sorbitol, maltitol, isomalt, mannitol, polyglycitol, lactitol, erythritol, hydrolyzed starch and combinations thereof;

Such a microbial stable emulsion, wherein the dispersant is a natural biopolymer selected from the group consisting of gum acacia, chemically modified gum acacia, starch, chemically modified starch, guar gum, locus bean gum, pectin, xanthan gum, carrageenan, konjac and combinations thereof;

Such a microbial stable emulsion, wherein the dispersed phase is selected from oleoresins, flavor oils, color oils, and oleoresin herbal extracts;

Such a microbial stable emulsion, wherein the flavor oil is selected from sautéed sweet onion flavor, garlic flavor and basil flavor oils;

Such a microbial stable emulsion, wherein the color oil is a capsicum oleoresin;

Such a microbial stable emulsion, which may be labelled natural;

Such a microbial stable emulsion, which is food grade;

Such a microbial stable emulsion, which is a paint, a cosmetic, a pharmaceutical, or a food;

A method of making a microbial stable emulsion, comprising selecting a lipophilic dispersed phase, selecting one or more dispersion medium characterized by low water activity, selecting one or more dispersants characterized by higher water binding capacity, and mixing the foregoing to form an emulsion which is microbially stable for at least twelve months;

Such a method of making a microbial stable emulsion, wherein the dispersant is selected for its ability to provide steric stabilization of the emulsion;

Such a method of making a microbial stable emulsion, wherein the dispersant is selected for its ability to provide steric stabilization of the emulsion through charge distribution.

### DETAILED DESCRIPTION OF THE INVENTION

Emulsions

The term “emulsion” when applied to the mixtures of the instant invention refers to a mixture that comprises a hydrophobic dispersed phase and dispersion media, the mixture being stable without microbial growth for minimum of twelve months.

The phrase “microbial stable”, as used in connection with the instant emulsions, refers to mixtures wherein no growth of bacteria, yeast, and fungi is observerd during the shelf life of the emulsion product.

Microbial stable emulsions are formed by lowering the water activity of such emulsions using dispersion medium exhibiting high water binding capacity and exhibiting structural conformations suited for optimized steric stabilizers. This method of controlling microbial growth is novel for requiring no antimicrobial agents, thereby permitting such emulsions to be labeled as natural.

The instant emulsions comprise dispersion media exhibiting low water activity. Such dispersion media may be selected from small molecular weight carbohydrates, water, ethanol, and biopolymers. Small molecular weight carbohydrates may be selected from glycerol, sugar, sugar alcohols and glucose oligomers selected from the group consisting of sucrose, glucose, fructose, maltose, lactose, galactose, xylose, xylitol, sorbitol, maltitol, isomalt, mannitol, polyglycitol, lactitol, erythritol, hydrolyzed starch and combinations thereof. Small molecular weight carbohydrates may be characterized by having a mass fraction of between about 0.2 and about 0.7. Small molecular weight carbohydrates may be characterized by having a mass fraction of between about 0.3 and about 0.7. Water may be characterized by having mass fraction of between about 0.1 and about 0.8. Ethanol may be characterized by having a mass fraction of between about 0.0 and about 0.4. Biopolymers may be selected from the group consisting of gum acacia, chemically modified gum acacia, starch, chemically modified starch, guar gum, locus bean gum, pectin, xanthan gum, carrageenan, konjac and combinations thereof. Biopolymers may be characterized by having a mass fraction of between about 0.001 and about 0.2.

The dispersed phase of the instant novel emulsions is selected for exhibiting structural conformation and charge distribution (zeta potential) suitable for properly interacting with steric stabilizing polymers.

### EXPERIMENTAL PART

The microbial stable emulsions of the present invention will be better understood in connection with the following examples, which are intended as an illustration of and not a limitation upon the scope of the invention.

### EXAMPLE OF REPRESENTATIVE PHARMACEUTICAL COMPOSITIONS

### Example 1.

Zeaxanthin Emulsion

| Active Ingredient: | Quantity |
|---|---|
| Zeaxanthin solution | 20% |
| Other Ingredients: | |
| Glycerol | 32% |
| Water | 24% |
| Ethanol | 14% |
| Gum acacia (steric stabilizer) | 10% |

1. Glycerol and water are mixed, then gum acacia is dissolved using a hand blender

2. Ethanol is added to the mixture

3. Zeaxanthin Oil is added to the dispersion medium

4. Shear mixed at 5000 rpm for 5 min. using a high shear mixer (Ross Mixer model HSM-100LSK, Hauppauge, N.Y.) at 5000 rpm for 5 min

### EMULSION EXAMPLES

The following examples are given by way of illustration only and are not to be construed as limiting.

### MICROBIALLY STABLE EMULSION (Food Applications)

### Example 1.

Sauteed Sweet Onion Flavor Emulsion

| Active Ingredient: | Quantity |
|---|---|
| Sauteed Sweet Onion Flavor Oil | 20% |
| Other Ingredients: | |
| Glycerol | 32% |
| Water | 24% |
| Ethanol | 16% |
| Gum acacia (steric stabilizer) | 8% |

1. Glycerol and water are mixed, then gum acacia is dissolved into the mixture using a hand blender

2. Ethanol is added to the mixture

3. Sautéed Sweet Onion Flavor Oil is added to the dispersion medium

4. Shear mixed at 5000 rpm for 5 min. using a high shear mixer (Ross Mixer model HSM-100LSK, Hauppauge, N.Y.) at 5000 rpm for 5 min.

### Example 2.

Garlic Flavor Emulsion

| Active Ingredient: | Quantity |
|---|---|
| Garlic Oil | 4% |
| Other Ingredients: | |
| Glycerol | 40% |
| Water | 32% |
| Ethanol | 14% |
| Gum acacia (steric stabilizer) | 10% |

1. Glycerol and water are mixed, then gum acacia is dissolved using a hand blender

2. Ethanol is added to the mixture

3. Garlic Oil is added to the dispersion medium

4. Shear mixed at 5000 rpm for 5 min. using a high shear mixer (Ross Mixer model HSM-100LSK, Hauppauge, N.Y.) at 5000 rpm for 5 min

### Example 3.

Basil Flavor Emulsion

| Active Ingredient: | Quantity |
|---|---|
| Basil Oil | 20% |
| Other Ingredients: | |
| Glycerol | 32% |
| Water | 24% |
| Ethanol | 14% |
| Gum acacia (steric stabilizer) | 10% |

1. Glycerol and water are mixed, then gum acacia is dissolved using a hand blender

2. Ethanol is added to the mixture

3. Basil Oil is added to the dispersion medium

4. Shear mixed at 5000 rpm for 5 min. using a high shear mixer (Ross Mixer model HSM-100LSK, Hauppauge, N.Y.) at 5000 rpm for 5 min

### Example 4.

Garlic Flavor Emulsion

| Active Ingredient: | Quantity |
|---|---|
| Garlic Oil | 40% |
| Other Ingredients: | |
| Glycerol | 25% |
| Water | 18% |
| Ethanol | 11% |
| Gum acacia (steric stabilizer) | 6% |

1. Glycerol and water are mixed, then gum acacia is dissolved using a hand blender

2. Ethanol is added to the mixture

3. Garlic Oil is added to the dispersion medium

4. Shear mixed at 5000 rpm for 5 min. using a high shear mixer (Ross Mixer model HSM-100LSK, Hauppauge, N.Y.) at 5000 rpm for 5 min

### MICROBIAL STABILITY

The microbial stable for emulsions of the instant invention and methods of preparing such, are characterized by unique and advantageous properties, rendering the “subject matter as a whole”, as claimed herein, unobvious. The microbial stable emulsions exhibit, in standard accepted reliable test procedures, the following valuable properties and characteristics:

### METHODS

Stability Assay

A microbial challenge test is performed on test emulsion using Aspergillus echinulatus ATCC 42687(minimum Aw=0.64) to evaluate whether a microorganism requiring low water activity is able to grow in an optimum growth temperature.

Moreover, emulsion stability is measured by observing the test emulsion over time for formation flocculation and resulting creaming, sedimention, or coalescence.

Objective:

The microbial stability of the test emulsion may be determined by preparing an emulsion comprising a test dispersed phase, such emulsion being free of antimicrobial agents. The test emulsion is inoculated with a challenge microorganism capable of growing in aqueous media characterized by low water activity (e.g. Aspergillus Echinulatus). The inoculated test emulsion is prepared in an oxygen permeable container and placed in an incubator maintained at the optimum growth temperature of the challenge microorganism. Time interval samples are taken for a period of 6 months. After suitable dilution using sterile water, samples are plated on an agar medium. After sufficient culture time in an incubator, the number of colonies is counted to calculate challenge microorganism concentration.

Test Material:

An emulsion comprising a flavor oil is prepared without the addition of antimicrobial agents.

Test Material Description:

The test emulsion is prepared by combining 20% flavor oil, 32% glycerol, 24% water, 16% ethanol, and 8% gum acacia. The gum acacia is first dissolved in water and glycerol. Ethanol is added to the mixture. After adding flavor oil, a high shear mixer is used to reduce the particle size of the dispersed phase to form a stable emulsion. The water activity of the dispersion medium is 0.61. Control emulsion is prepared by combining in a similar fashion 20% flavor oil with water as the dispersion medium.

Procedure:

Triangle sensory tests are performed to evaluate test emulsion flavor with that of the the control emulsion. The emulsions are filled in oxygen permeable containers and then inoculated with Aspergillus echinulatus ATCC 42687. Innoculated test and control emulsion are incubated at 30-35° C. to measure microbial concentration as a function of time. At the same time the phase stability of the test and control emulsions is monitored.

Results:

The triangle sensory test shows no difference in flavor between the test emulsion and the control emulsion. The control emulsion shows an increase in concentration of the challenge microorganism in contrast to the test emulsion which shows no increase in challenge microorganism concentration. Both the test and control emulsions are stable during the test period of six months.

Conclusions:

The test emulsion comprising a low water activity dispersion medium prevents growth of fungi, which microorganism infestation requires the lowest water activity to grow among test microorganisms. Moreover, the test emulsion is stable for six months. Sensory tests show that the test emulsion has no effect on the organoleptic quality of the dispersed phase flavor versus the control emulsion.

### CONCLUSIONS

In conclusion, from the foregoing, it is apparent that the present invention provides novel, valuable, and unpredictable applications and uses of the microbial stable emulsions of the present invention, which microbial stable emulsions do not comprise microbial stabilizers. Compositions prepared therewith demonstrate more specifically-enumerated characteristics and advantages.

The present invention is not to be limited in scope by the specific embodiments described herein. Indeed, various modifications of the invention in addition to those described herein will become apparent to those skilled in the art from the foregoing description.

All patents, applications, publications, test methods, literature, and other materials cited herein are hereby incorporated by reference.

We claim:

1. A microbial stable emulsion comprising dispersion media characterized by lower water activity, one or more dispersants characterized by higher water binding capacity, and a lipophilic dispersed phase, wherein the dispersion media is present at a concentration below that in which microorganisms may grow in the emulsion.

2. The microbial stable emulsion of claim 1 which does not comprise an antimicrobial agent.

3. The microbial stable emulsion of claim 1 which is characterized by being resistant to microorganism contamination, wherein such microorganism contamination is selected from bacterial, yeast and fungal contamination.

4. The microbial stable emulsion of claim 1, which is characterized by being resistant to microbial growth for a period exceeding twelve months.

5. The microbial stable emulsion of claim 1, which is characterized by being resistant to microbial growth for a period exceeding four months.

6. The microbial stable emulsion of claim 1, which is characterized by being resistant to microbial growth for a period exceeding two months.

7. The microbial stable emulsion of claim 1, wherein the dispersion media may be characterized by a water activity below 0.95.

8. The microbial stable emulsion of claim 1, wherein the dispersion media comprises one or more small molecular weight carbohydrates, water, ethanol.

9. The microbial stable emulsion of claim 8, wherein the dispersion media small molecular weight carbohydrate may be selected from glycerol, sugar, sugar alcohols and glucose oligomers selected from the group consisting of sucrose, glucose, fructose, maltose, lactose, galactose, xylose, xylitol, sorbitol, maltitol, isomalt, mannitol, polyglycitol, lactitol, erythritol, hydrolyzed starch and combinations thereof.

10. The microbial stable emulsion of claim 1, wherein the dispersant is a natural biopolymer selected from the group consisting of gum acacia, chemically modified gum acacia, starch, chemically modified starch, guar gum, locus bean gum, pectin, xanthan gum, carrageenan, konjac and combinations thereof.

11. The microbial stable emulsion of claim 1, wherein the dispersed phase is selected from oleoresins, flavor oils, color oils, and oleoresin herbal extracts.

12. The microbial stable emulsion of claim 11, wherein the flavor oil is selected from sautéed sweet onion flavor, garlic flavor and basil flavor oils.

13. The microbial stable emulsion of claim 11, wherein the color oil is a capsicum oleoresin.

14. The microbial stable emulsion of claim 1, which may be labelled natural.

15. The microbial stable emulsion of claim 1, which is food grade.

16. The microbial stable emulsion of claim 1, which is a paint, a cosmetic, a pharmaceutical, or a food.

17. A method of making a microbial stable emulsion, comprising selecting a lipophilic dispersed phase, selecting one or more dispersion medium characterized by low water activity, selecting one or more dispersants characterized by higher water binding capacity, and mixing the foregoing to form an emulsion which is microbially stable for at least six months.

18. The method of making a microbial stable emulsion of claim 17, wherein the dispersant is selected for its ability to provide steric stabilization of the emulsion.

19. The method of making a microbial stable emulsion of claim 18, wherein the dispersant is selected for its ability to provide steric stabilization of the emulsion through charge distribution.
